(19) United States
(12) Patent Application Publication
(10) Pub. No.: US 2007/0179348 A1
(43) Pub. Date: Aug. 2, 2007

(54) Biochip scanning device for portable electronic products

(76) Inventor: Ein-Yiao Shen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/211,680

(22) Filed: Aug. 26, 2005

(51) Int. Cl. A61B 5/00; G06K 7/00

(52) U.S. Cl. 600/300; 235/435

(57) ABSTRACT

A biochip scanning device for a portable electronic product at least comprises a signal scanning module, a signal processing module and a data processing module; said signal scanning module scanning and reading a first signal of a biochip disposed on a surface, or under a skin or inside a body, said signal processing module receiving and processing said first signal and sending a second signal to said data processing module for obtaining a third signal; said third signal driving said portable electronic product to provide display, alert, storage and remote transmission functions; said biochip scanning device facilitating and simplifying processing of an output signal of said biochip disposed on a surface, under a skin or inside a body, besides, said biochip scanning device enhancing the functionality of said portable electronic product.

### BACKGROUND OF THE INVENTION

1. Field of the Invention

The present invention is related to a biochip scanning device for a portable electronic product, and more particularly, to a biochip scanning device capable of scanning and reading signals of biochips to enable the portable electronic product to provide display, alert, storage and remote transmission functions.

2. Description of the Prior Art

As technology evolving, various portable electronic products (e.g. mobile phone, personal digital assistant, and palmtop) are making strides towards providing versatile functions. For example, mobile phone evolves from a sheer voice reception/transmission gadget to a device combining web browsing, digital photography, GPS, voice recording, music playback and many other functions. As the popularity of portable electronic products keeps rising and almost everyone has one in hand now, it is worthwhile to exploit this characteristic and add more features to portable electronic products. Nowadays, surface detection methods for measuring body temperature, blood pressure, pulse, blood sugar, oxygen saturation in blood have been widely adopted and many portable biological condition detecting/monitoring systems have been matured. Therefore, if it's possible to use data detected by sensors to enable mobile phones to provide display, alert, storage and transmission functions, it will create much more benefit for mankind.

Due to the rapid development of biochips and biological condition detection technologies, many changes of one's biological conditions can be monitored by biochip attached to body surface or embedded inside one's body and used to determine if he/she is ill. Then he/she can quickly find out what's wrong with his/her body. Recently there has been much progress in the study of biotech, aside from the booming development of biochips, the development of microelectronic devices and nanometer technology also accelerates the trend of using devices on the body surface, inside the body, or even swallowed by mouth and sent to digestive organs, to detect biological data. Furthermore, the availability of radio frequency identification (RFID) also makes it possible to combine biochips with RFID technology to facilitate signal-reading process.

As mentioned above, how to leverage the highly prevailing portable electronic products to monitor variations of biological conditions to provide health care, is one of the most important guidelines to develop portable electronic products in the future.

Therefore, the two devices mentioned above present several shortcomings to be overcome.

In view of the above-described deficiency of prior-art seals, after years of constant effort in research, the inventor of this invention has consequently developed and proposed a biochip scanning device for portable electronic products.

### SUMMARY OF THE INVENTION

It is an object of the present invention to provide a biochip scanning device for a portable electronic product, using a signal scanning module to read data stored on a biochip and to enable the portable electronic product to provide display, alert, storage and remote transmission functions

It is another object of the present invention to provide a biochip scanning device for a portable electronic product, the biochip scanning device working with biochips, surface detection devices, portable devices, or any other biological condition detection devices embedded with RFID or similar bar-code technologies.

It is another object of the present invention to provide a biochip scanning device, which is small in size to be miniaturized into a chip or a module scale and easy to operate, and widely applicable to various applications.

The biochip scanning device for a portable electronic product at least comprises a signal scanning module, a signal processing module and a data processing module; the signal scanning module scanning and reading a first signal of a biochip disposed on a surface or under a skin or inside a body, the signal processing module receiving and processing the first signal and sending a second signal to the data processing module for obtaining a third signal; the third signal driving the portable electronic product to provide display, alert, storage and remote transmission functions; the biochip scanning device facilitating and simplifying processing of an output signal of the biochip disposed on a surface or under a skin or inside a body, besides, the biochip scanning device enhancing the functionality of the portable electronic product.

### BRIEF DESCRIPTION OF THE DRAWINGS

FIG. 1 illustrates a block diagram of the biochip scanning device for portable electronic products;

FIG. 2A illustrates a first embodiment of the biochip scanning device for portable electronic products;

FIG. 2B illustrates a second embodiment of the biochip scanning device for portable electronic products; and

FIG. 2C illustrates a third embodiment of the biochip scanning device for portable electronic products.

### DETAILED DESCRIPTION OF THE PREFERRED EMBODIMENT

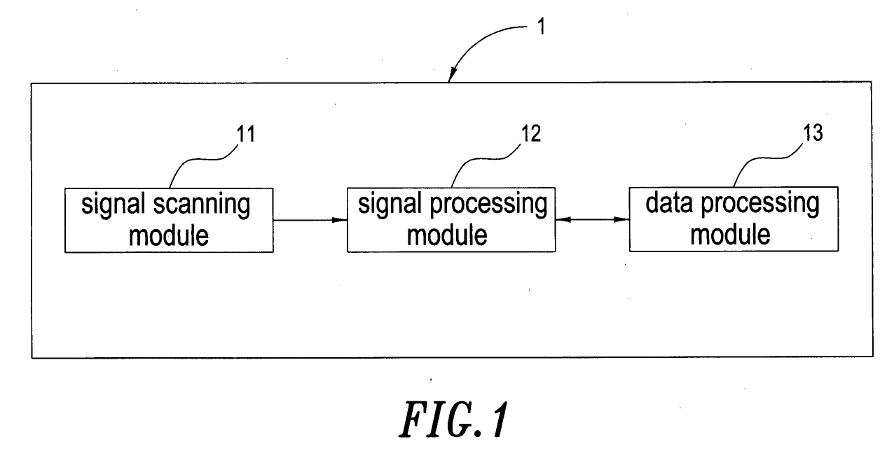

Please refer to FIG. 1, which illustrates a block diagram of the biochip scanning device for portable electronic products, wherein the biochip scanning device is the biochip scanning device 1 connected to portable electronic product, at lease comprises:

A signal scanning module 11 for scanning and reading a detected signal containing data of a sensor (e.g. biochip, surface detection device, portable detector, or any other biological detection devices) and outputting the detected signal to a signal processing module 2;

A signal processing module 12 for converting the detected signal from signal scanning module 11 into a data signal and sending the data signal to a data processing module 13;

Data processing module 13 for analyzing and storing the data signal from signal processing module 12 and obtaining a result, data processing module 13 outputting the result to a function module of the portable electronic product;

When signal scanning module 11 scans and reads the detected signal coming from outside, the signal processing module 12 converting the detected signal into the data signal, data processing module 13 analyzing and storing the data signal to obtain a result to drive the portable electronic product to provide display, alert, storage and remote transmission functions to monitor a biological condition of a human body.

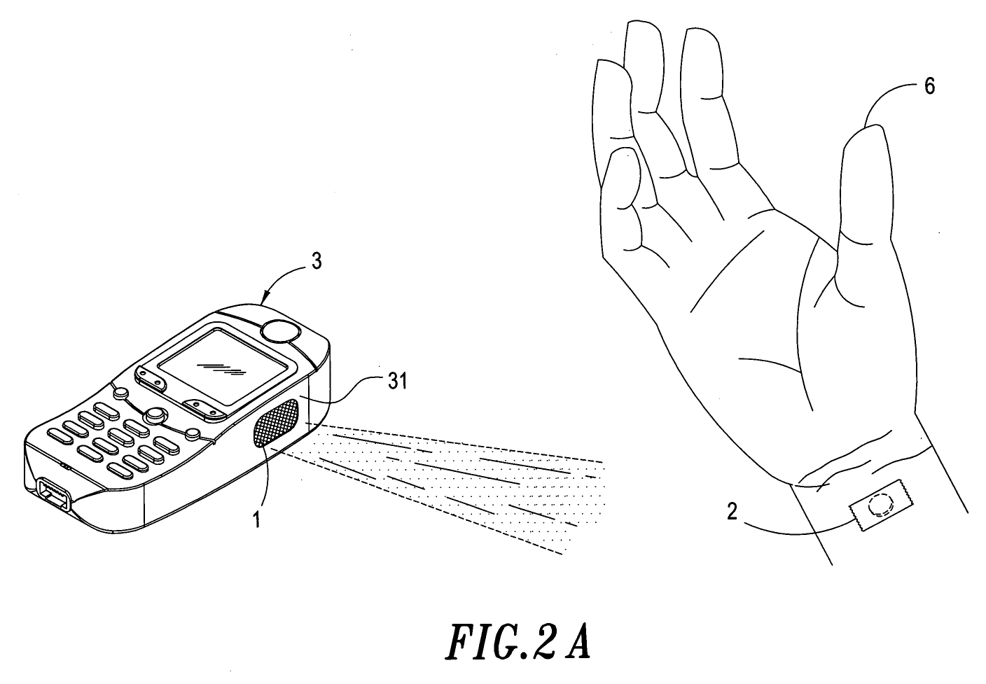

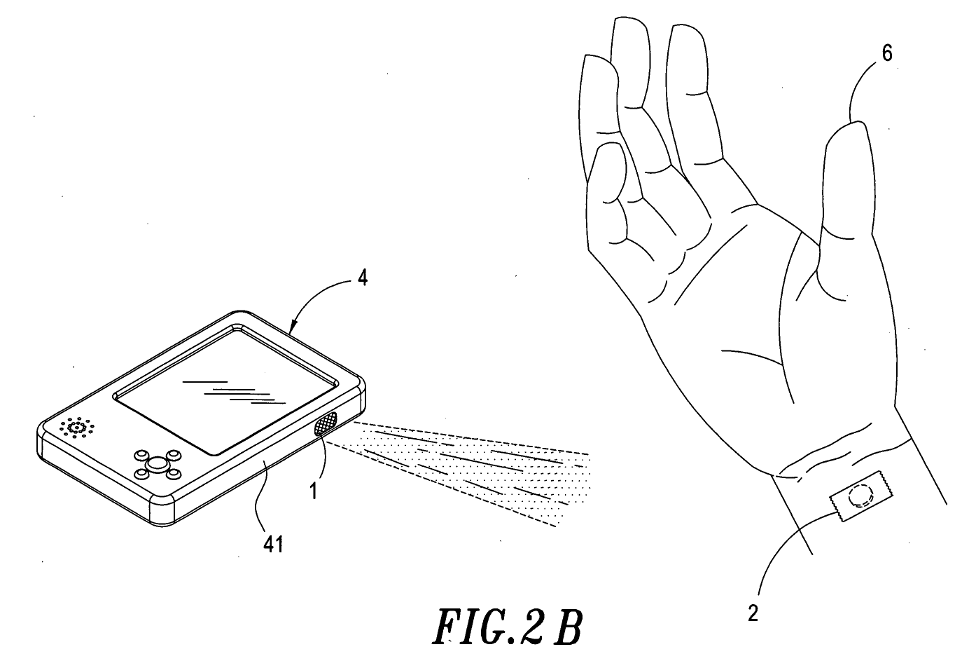

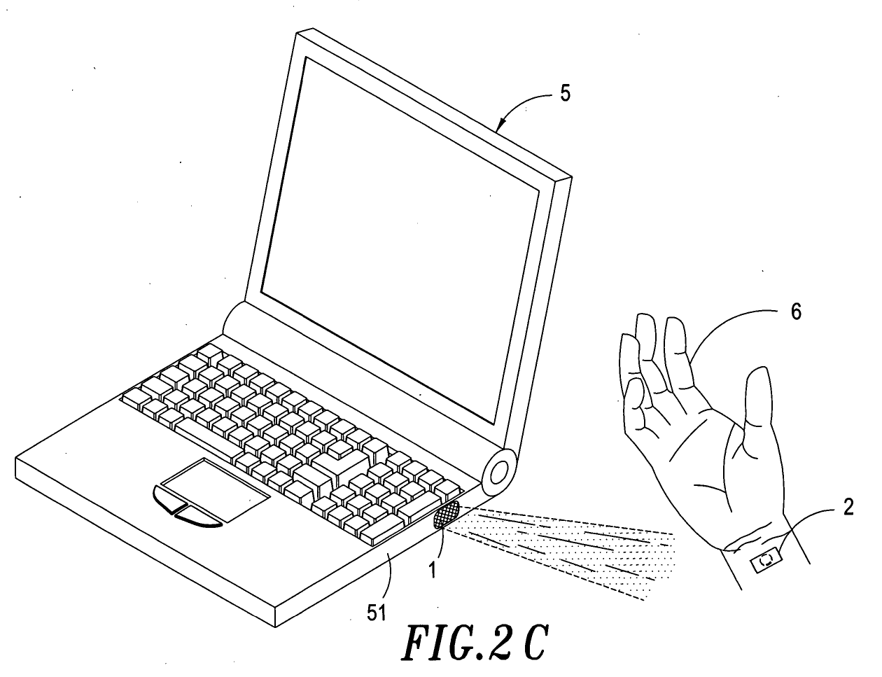

Please refer to FIG. 2A, which illustrates a first embodiment of the biochip scanning device for portable electronic products in the present invention, wherein the present invention can be placed inside a mobile phone 3, using biochip scanning device 1 exposed outside a case 31 to scan and read a signal containing biological condition data provided by a biochip 2 embedded with RFID technology on a user 6, then using mobile phone 3 to provide display, alert, storage and remote transmission functions.

Please refer to FIG. 2B, which illustrates a second embodiment of the biochip scanning device for portable electronic products in the present invention, wherein the present invention can be placed inside a personal digital assistant (PDA) 4, using biochip scanning device 1 exposed outside a case 41 to scan and read a signal containing biological condition data provided by biochip 2 embedded with RFID technology on user 6, then using personal digital assistant (PDA) 4 to provide display, alert, storage and remote transmission functions.

Please refer to FIG. 2C, which illustrates a third embodiment of the biochip scanning device for portable electronic products in the present invention, wherein the present invention can be placed inside a notebook 5 or a palmtop, using biochip scanning device 1 exposed outside a case 51 to scan and read a signal containing biological condition data provided by biochip 2 embedded with RFID technology on user 6, then using notebook 5 to provide display, alert, storage and remote transmission functions.

The present invention can be implemented as an IC chip, which is easy to manufacture and can be placed inside the portable electronic product.

The biochip scanning device for portable electronic products, compared with prior art techniques, is advantageous in:

1. The present invention provides a biochip scanning device for a portable electronic product, using a signal scanning module to read data stored on a biochip and to enable the portable electronic product to provide display, alert, storage and remote transmission functions. In other words, the biochip scanning device creates added value for the portable electronic product.

The present invention provides a biochip scanning device for a portable electronic product, the biochip scanning device can work with biochips, surface detection devices, portable devices, or any other biological condition detection devices embedded with RFID or similar bar-code technologies.

The present invention provides a biochip scanning device, which is small in size to be miniaturized into a chip or a module scale and easy to operate, and widely applicable to various applications.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A biochip scanning device for portable electronic product, at least comprises:
a signal scanning module for scanning and reading a first signal containing data of a sensor, said signal scanning module outputting said first signal to a signal processing module;
said signal processing module for converting said first signal from said signal scanning module into a second signal and sending said second signal to a data processing module;
said data processing module for analyzing and storing said second signal from signal processing module and obtaining a third signal, said data processing module outputting said third signal to a function module of said portable electronic product; and
when said signal scanning module scanning and reading said first signal coming from outside, said signal processing module converting said first signal into said second signal, said data processing module analyzing and storing said second signal to obtain said third signal to drive said portable electronic product to provide display, alert, storage and remote transmission functions to monitor a biological condition of a human body.

2. T he biochip scanning device of claim 1, wherein said sensor is a biochip.

3. The biochip scanning device of claim 1, wherein said sensor is a surface detection device.

4. The biochip scanning device of claim 1, wherein said sensor is a portable detector.

5. The biochip scanning device of claim 1, wherein said sensor is any one of biological detection devices.

6. The biochip scanning device of claim 1, wherein said portable electronic product is a mobile phone.

7. The biochip scanning device of claim 1, wherein said portable electronic product is a personal digital assistant (PDA).

8. The biochip scanning device of claim 1, wherein said portable electronic product is a notebook PC or a handheld PC.
